(19) United States
(12) Patent Application Publication
(10) Pub. No.: US 2012/0029223 A1
(43) Pub. Date: Feb. 2, 2012

(54) METHOD FOR PRODUCTION OF SUBSTITUTED ALKYL MALONIC ESTERS AND DERIVATIVES THEREOF

(76) Inventors: Gad Friedman, Rehovot (IL); Andreas Stadelmaier, Mutlangen (DE)

(21) Appl. No.: 12/844,338

(22) Filed: Jul. 27, 2010

(51) Int. Cl. C07C 303/30

(52) U.S. Cl. 558/52

(57) ABSTRACT

Substituted alkyl methyl malonate compounds are produced in an essentially one step method, the method suitable for large scale production of alkyl malonate compounds. According to one embodiment the method comprises reacting a methyl malonate with a di-functional, doubly tosylated, alkyl chain.

### FIELD OF THE INVENTION

The invention relates to the production of substituted alkyl methyl- or lower alkyl-malonates. In one example the invention relates to the production of 2-(5-tosylpentyl)-2-methyl malonic acid derivatives.

### BACKGROUND OF THE INVENTION

2-(5 ¹⁸fluoro pentyl)-2-methyl malonic acid belongs to a family of low-molecular-weight compounds used for the imaging of apoptosis in vivo.

Apoptosis is a controlled program of cell death that is inherent in every cell and which plays important roles in normal tissue homeostasis and in the etiology or pathogenesis of numerous medical disorders. Molecular imaging of this process in clinical practice may thus enhance diagnosis, monitoring of disease course and monitoring of the efficacy of treatment for a wide array of diseases, including myocardial infarction, cerebral stroke and cancer.

Alkyl methyl malonic acid compounds respond to the alterations in plasma membrane potential and phospholipid scrambling, which are hallmarks of apoptotic cells. Systemically administered, these compounds cross the intact plasma membrane of apoptotic cells and accumulate in the cytoplasm, enabling detection of apoptotic cells from the early stages of the cell death process.

¹⁸F-labeled 2-(5-fluoropentyl)-2-methyl-malonic acid (also termed ¹⁸F-ML-10) is an example of a compact structured compound having a minimal number of functional groups, harboring a radioisotope, specifically designed to meet the challenges of clinical imaging of apoptosis by PET.

A common route for the preparation of malonic esters, commonly known as “malonic synthesis”, is demonstrated in Scheme A. This route includes two consecutive steps of reactions in which a deprotonated dialkyl malonate compound is reacted with an alkyl possessing a good leaving group, then, after isolation and purification, a second deprotonation with the aid of a strong base takes place, followed by addition of a second alkyl having a good leaving group and possibly with a (protected) functional moiety, to obtain a desired dialkyl malonate. The sequence of the alkylations may be interchanged.

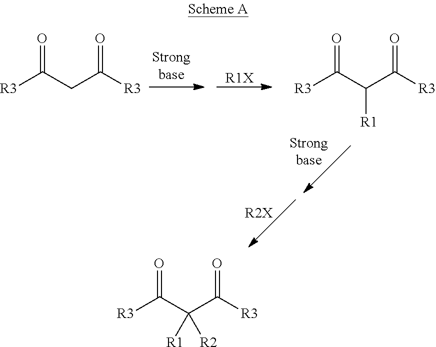

R1=alkyl;

R2=alkyl (possibly with a protected functional moiety);

R3=lower alkyl;

Strong base=e.g. NaOMe, NaH or KHMDS; and

X=leaving group such as Br, Cl, sulfate, tosylate or mesylate.

Scheme B illustrates a known method for producing an alkyl malonate, namely, 2-(4-fluorobutyl)-2-methyl-malonate, which starts by the protection of a hydroxyl bromo alkyl chain (1) and deprotonation of diethylmethyl malonate with a strong base. The deprotonated malonate is reacted with the protected hydroxyl bromo alkyl chain (2) to obtain a protected hydroxyl alkyl diethylmethyl malonate compound (3). After a deprotection step, the resulting intermediate (4) is reacted with methanesulfonyl chloride (mesyl chloride) to obtain a mesylated precursor (5) which serves as a good precursor for fluorination and/or radio-labeling.

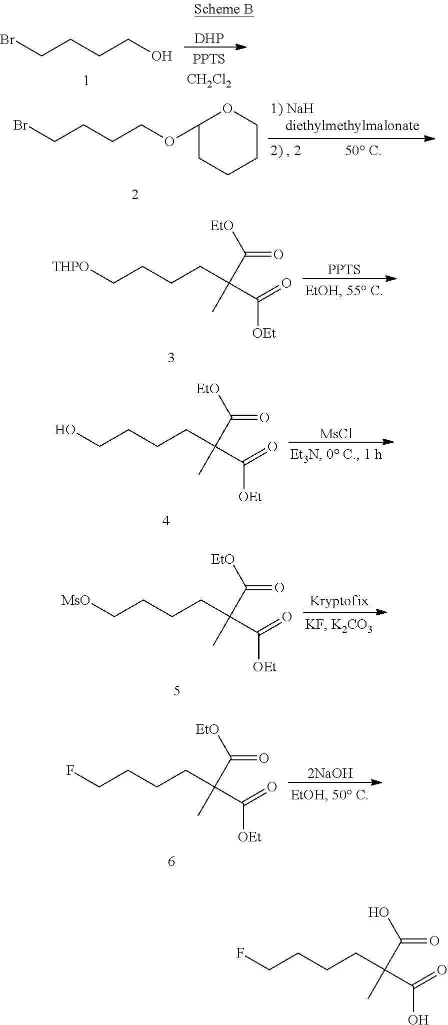

Known production methods for alkyl methyl malonates, which employ multiple steps, including auxilary protection and deprotection steps, may render these methods expensive and impractical for large scale production, especially so when GMP protocol is considered.

### SUMMARY OF THE INVENTION

Embodiments of the invention provide a novel and simple method for the production of substituted alkyl malonate compounds, for example, alkyl methyl malonate compounds and their homologues and both structural and functional analogs.

According to embodiments of the invention substituted alkyl methyl malonate compounds are produced in an essentially one step high yield method, providing a method suitable for large scale production of alkyl malonate compounds.

According to one embodiment the method comprises reacting a methyl malonate with a di-functional, doubly tosylated alkyl chain.

According to one embodiment there is provided a method for the preparation of a substituted alkyl methyl malonic ester compound of Formula III

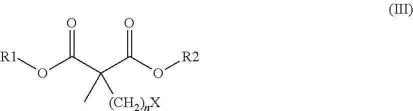

the method comprising reacting a 2-methyl malonic ester of Formula I

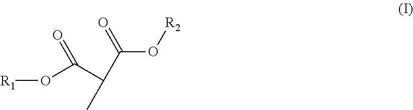

with a di-substituted alkyl of Formula II:

X(CH₂)n-X (II)

wherein R₁ and R₂ are each independently a C₁₋₈ linear or branched alkyl;

n=2-18; and X is a leaving group.

According to some embodiments R₁ and R₂ are each independently methyl, ethyl, propyl, isopropyl, tert-butyl or benzyl. According to one embodiment R₁ and R₂ are each tert-butyl.

According to another embodiment n is 5. According to another embodiment X is a sulfonate, such as tosylate.

According to one embodiment R₁ and R₂ are each tert-butyl; n is 5; and X is a tosylate.

The step of reacting the 2-methyl malonic ester of Formula I with the di-substituted alkyl of Formula II may be carried out in the presence of a base. According to one embodiment the base is a strong base.

The method may further include a step of crystallizing out excess reagents.

According to some embodiments, the compound of Formula II is added to the reaction in excess, (according to some embodiments four-fold excess or more) over the compound of Formula I.

According to one embodiment the step of reacting the compound of Formula II with the compound of Formula I is under dry conditions, such as in an inert atmosphere.

According to one embodiment reacting the compound of Formula II with the compound of Formula I is at a temperature between room temperature and reflux temperature. According to one embodiment reacting the compound of Formula II with the compound of Formula I is at about 50° C.

According to one embodiment there is provided a 2-(5-tosylpentyl)-2-methyl di-tert butyl malonic acid compound the crystallized form of which has a melting point of 36.5-38.5° C.

According to additional embodiments there is provided a 2-(5-tosylpentyl)-2-methyl di-tert butyl malonic acid compound having the mass spectrometry, ¹H-NMR, ¹³C-NMR and IR results as described below.

### DETAILED DESCRIPTION OF THE INVENTION

According to one embodiment the method comprises reacting a 2-methyl malonate with an alkyl chain, symmetrically substituted with two leaving groups, and adding a strong base to obtain an alkyl methyl malonate.

Scheme 1 illustrates a synthetic scheme according to one embodiment of the invention.

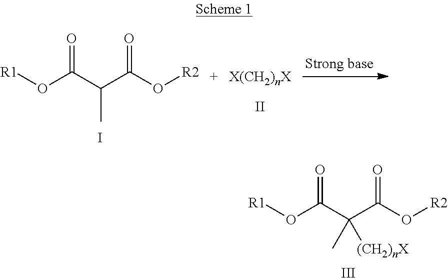

In the method according to one embodiment, a methyl malonate (compound I, in which R₁ and R₂ each independently represent a branched or unbranched C₁₋₈ alkyl) is deprotonated, due to the presence of the strong base, to act as a nucleophile to attack compound II in which n=2-18 and X represents a leaving group.

Potentially, the nucleophilic attack can happen at both ends of compound II, resulting in the formation of dimers (see compound IV below).

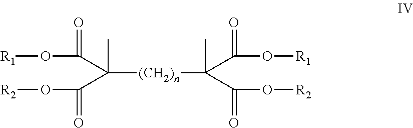

Furthermore, the reaction of de-protonated malonate with substituted alkyl is exothermic so, in order to avoid a high temperature environment, substituted alkyl is usually added slowly and gradually. In this case, the local concentration of malonate in the reaction mixture may be higher than the concentration of substituted alkyl, especially during the initial steps of the process, favoring dimer formation.

To avoid dimer formation, methods according to embodiments of the invention start out with an excess of a di-functional substituted alkyl (compound II). This way, for each de-protonated malonate formed with the addition of a strong base, there is a surplus of substituted alkyl compound, statistically favoring the formation of monomers rather than dimers.

According to one embodiment a fourfold molar excess of substituted alkyl may be used.

The group X (in compound II) may be a suitable leaving group. For example, X may be a sulfonate (e.g., tosylate, mesylate, nosylate or brosylate), a phenyl compound having a nitro group or a halogen, such as bromide.

The symmetrical two leaving groups are typically chosen so that their reactivity is suitable for the malonic synthesis, and matches the reactivity required for the efficient preparation of the final product (e.g. fluoride formation), yet not compromising the stability of the product.

The base used according to embodiments of the invention may be any suitable base, such as any of the strong bases NaH, NaOMe, NaOEt or KHMDS. Other bases may be used. According to one embodiment the base is used in a ratio of base/compound I in between 1.0 to 1.5.

Scheme 2 illustrates the preparation of a 2-(5 substituted-alkyl)-2-methyl malonic acid derivative. Methods according to embodiments of the invention will be exemplified by showing the preparation of a 2-(5-tosyl pentyl)-2-methyl malonate, however other substitutions may be similarly prepared.

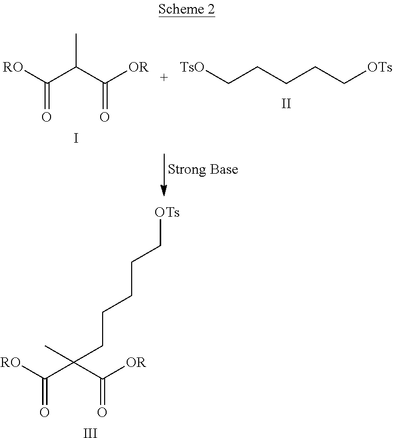

R is a protecting group such as methyl, ethyl, propyl, isopropyl, tert-butyl, benzyl or low alkyls. Other protecting groups may be used.

According to one embodiment, compound II is added in excess thus the synthesis according to the example shown in Scheme 2 may end with a large excess of the di-functional intermediate II. Excess reagents such as compound II may be crystallized out of the solution in an initial purification step (subsequent purification steps may be also employed). The purification step may be easier to achieve with di-functional intermediate compounds having high melting points. Compound III, on the other hand, has a typically lower melting point in solution (according to one embodiment the melting point of Compound III is 38.5° C.). Thus, carrying out the crystallization, at temperatures in which the low melting compound III is still in solution, may be advantageous.

Compound II, in the example shown in Scheme 2 is a ditosylate having a high melting point (96° C.).

Tosylates, brosylates and nosylates are examples of di-functional substituted compounds that can enable efficient crystallization of compound II from the reaction mixture at the initial purification step.

For example, Table 1 demonstrates known melting points of several ditosylates, which may be used in embodiments of the invention. Other suitable tosylates may be used.

TABLE 1

| | Ditosylate | |
|---|---|---|
| Parent Alcohols | M.P. ° C. | Crystallization Solvent |
| Ethylene Glycol | 124-127 | Methanol |
| Diethylene Glycol | 87-87.5 | Methanol |
| 1,3-Propanediol | 89-92 | Methanol |
| 1,4-Butanediol | 82-83 | Methanol |
| 1,5-Pentanediol | 96 | Methanol |
| 1,6-Hexanediol | 77-78 | Ethanol |

According to embodiments of the invention, the product of tosylate replacement of compound III (in both Scheme 1 and Scheme 2) may be hydrolyzed to produce a malonic acid compound. Hydrolysis may take place in an alkali or acidic environment or in any other suitable reaction, as known in the art.

According to embodiments of the invention, compound III (in both Scheme 1 and Scheme 2) may be labeled to obtain, for example, a marker of apoptosis. According to one embodiment compound III may be labeled by ¹⁸F. Other radioactive labels, such as ³H may be used. For example, radio-isotopes of the metal ions Tc, oxo-Tc, In, Cu, Ga, Xe, Tl and Re, oxo-Re may be used for radio-isotope scans such as SPECT; Gd (III), Fe (III) or Mn (II) for MRI; and ¹⁸F, ¹⁵O, ¹⁸O, ¹¹C, ¹³C, ¹²⁴I, ¹³N, ⁷⁵Br for positron emission tomography (PET) scan and ⁹⁰Y, ¹¹¹In, ¹⁷⁷Lu for radio therapeutic treatments.

A method for labeling may be used as known in the art (for example, see Reshef et al. Journal of Nuclear Medicine, 49(9), 1520-1528, 2008 and Panwar et. al. Cancer Biology & therapy, 4 (8) 854-860, 2005).

According to some embodiments of the invention, common complexants of radioisotopes like Y, Ir Lu-DOTA (1,2,7,10 tetra-azacyclododecane N,N′,N″,N″″ tetra-acetic acid) can be attached to compound III, with the aid of an amine terminated linker (e.g., see Panwar et al.).

Some examples will now be described to further illustrate the invention and to demonstrate how embodiments of the invention may be carried-out in practice.

### EXAMPLES

### Example 1

Methyl di-tert-butyl malonate (18 g, 78.16 mmol) was dissolved, under an argon atmosphere, in 600 ml DMF and cooled to 0° C. KHMDS (157 ml; 0.5M in toluene) was added to this solution dropwise. The reaction mixture was allowed to reach room temperature and was stirred further for 2 hours. 1,5-Bis(p-tolylsulfonato) pentane (1.97 g, 235 mmol, 3 eq) was separately dissolved under an argon atmosphere in 100 ml dry DMF at room temperature. The KHMDS deprotonated malonate solution was added, drop-wise, over a period of 3 hours to the ditosylate solution. After completion, the reaction mixture was stirred for 72 hrs at RT, under inert atmosphere. The mixture was diluted with water (250 ml), extracted with diethyl ether (3×50 ml) and the combined organic phases were washed with brine, dried over a MgSO₄, filter and evaporated under reduced pressure. The oily residue was taken up in 200 ml methanol and cooled to 5° C. over night. The ditosylate that crystallized out was collected by filtration.

The mother liquor was evaporated, and the residue chromatographed on silica gel (eluent—Petroleum ether/ethyl acetate 10:1) to afford 9.4 g of syrup. HPLC showed that the syrup consisted of 75% of the target compound. The total calculated yield was 19.5%.

### Example 2

2-methyl di-tert butyl malonate (45 g, 0.2 mol) and 1,5 bis(p-toylsulfonato) pentane (322 g, 0.78 mol, 4 eq) were dissolved, under an argon atmosphere, in 1.6 L dry tetrahydrofuran (THF). Sodium hydride (NaH) (5.6 g, 1.2 eq) was added in one portion. The reaction mixture was heated up to 50° C. and stirring was maintained for 16 hrs at this temp. The mixture was filtered off, evaporated to dryness, diluted with tert-butyl methyl ether (TBME, 200 ml), filtered and evaporated again. The residue was taken up in TBME (100 ml) and was cooled in the refrigerator. The ditosylate compound crystallized out and was filtered off. The filtrate was evaporated to dryness affording (89 g, 97%) of the crude material as oil which solidified upon storage. Final purification was achieved by filtering the material through silica gel in a fritted funnel (Petroleum Ether/Ethyl Acetate 15:1/4:1) and further crystallization from ethanol to afford 50.5 g (57%) of a white pure solid having a melting point of 36.5-38.5° C.

Mass spectrometry (Finnigan Surveyor MSQ Plus (APCl, neg.) results of the 2-(5 substituted-alkyl)-2-methyl malonic acid derivative showed m/z=469.3 [M-H]⁻.

¹H-NMR [Bruker Avance 400 (400 MHz, CDCl₃, TMS as internal standard] of the 2-(5 substituted-alkyl)-2-methyl malonic acid derivative showed the following results: δ(ppm)=1.09-1.23 (m, 2H), 1.26 (s, 3H, Me), 1.27-1.36 (m, 2H), 1.43 (s, 18H, tBu), 1.57-1.74 (m, 4H), 2.45 (s, 3H, Me), 4.01 (t, 2H, j=6.5 Hz, CH₂), 7.34 (d, 2H, J=8.1 Hz, Ph) 7.78 (d, 2H, J=8.3 Hz, Ph).

¹³C-NMR [Bruker Avance 400 (100.6 MHz, CDCl₃, TMS as internal standard] of the 2-(5 substituted-alkyl)-2-methyl malonic acid derivative showed the following results: δ(ppm)=19.7, 21.6, 23.6, 25.8, 27.9 (6C), 28.6, 36.1, 54.5, 70.4, 80.9 (2C), 127.9 (2C), 129.9 (2C), 133.3, 144.7, 171.7 (2C).

IR (Bio-Rad FTS 3000MX (KBr)) results of the 2-(5 substituted-alkyl)-2-methyl malonic acid derivative showed: ν (cm⁻¹)=3454 (w, br), 3005 (w), 2990 (w), 2973 (m), 2952 (m), 2935 (m), 2868 (w), 1748 (m), 1725 (s), 1599 (w), 1466 (m), 1394 (w), 1371 (m), 1358 (m), 1309 (m), 1292 (m), 1278 (m), 1255 (m), 1239 (m), 1221 (w), 1179 (vs), 1156 (m), 1123 (m), 1119 (m), 1098 (m), 1043 (w), 1019 (w), 970 (m), 946 (m), 920 (w), 904 (m), 867 (w), 851 (w), 829 (m), 811 (m), 767 (m), 725 (w), 706 (w), 666 (m), 579 (m), 555 (m), 506 (w), 486 (w), 469 (w).

### Example 3

Aminophenyl alanine DOTA (1 equivalent) may be added to a stirred solution of compound III in THF, followed by the addition of triethyl amine (1.2 equivalent).

The reaction may be continued until compound III is consumed. THF may be evaporated. Water may be added and the pH adjusted to ˜5. The resulting mixture may be extracted with ether, washed with brine, dried and evaporated.

Purification by chromatography will provide the conjugate of malonate and DOTA. Similarly DOTA conjugates of methyl, ethyl and isopropyl ester analogs of compound III can be prepared.

The methods according to embodiments of the invention provide a substituted alkyl methyl malonate compound using minimal steps enabling a cleaner, high yield product and precursor for labeling that may be advantageously used in molecular imaging procedures using, for example, x-ray, CT scan, magnetic resonance imaging (MRI) or radio-isotope scans such as single photon emission tomography (SPECT) or positron emission tomography (PET), as well as in radioisotope therapy.

1. A method for the preparation of a substituted alkyl methyl malonic ester compound of Formula III 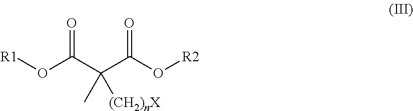
the method comprising reacting a 2-methyl malonic ester of Formula I 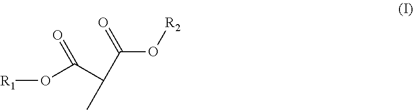
with a di-substituted alkyl of Formula II:
X(CH₂)n-X (II)
wherein R₁ and R₂ are each independently a C₁₋₈ linear or branched alkyl;
n=2-18; and
X is a leaving group.

2. The method according to claim 1 comprising reacting the 2-methyl malonic ester of Formula I with the di-substituted alkyl of Formula II in the presence of a strong base.

3. The method according to claim 1 comprising a step of crystallizing out excess reagents.

4. The method according to claim 1 wherein R₁ and R₂ are each independently selected from methyl, ethyl, propyl, isopropyl, tert-butyl or benzyl.

5. The method according to claim 4 wherein R₁ and R₂ are each tert-butyl.

6. The method according to claim 1 wherein n=5.

7. The method according to claim 1 wherein X is a sulfonate.

8. The method according to claim 7 wherein X is a tosylate.

9. The method according to claim 1 wherein
R₁ and R₂ are each tert-butyl;
n=5; and
X is a tosylate.

10. The method according to claim 1 comprising adding an excess of the compound of Formula II.

11. The method according to claim 10 comprising adding an excess of four-fold Formula II.

12. The method according to claim 1 wherein the step of reacting the compound of Formula II with the compound of Formula I is under dry conditions.

13. The method according to claim 12 wherein the step of reacting the compound of Formula II with the compound of Formula I is under an inert atmosphere.

14. The method according to claim 1 comprising reacting the compound of Formula II with the compound of Formula I at a temperature between room temperature and reflux temperature.

15. The method according to claim 14 comprising reacting the compound of Formula II with the compound of Formula I at about 50° C.

16. A 2-(5-tosylpentyl)-2-methyl di-tert butyl malonic acid compound the crystallized form of which has a melting point of 36.5-38.5° C.
